(19) United States
(12) Patent Application Publication
(10) Pub. No.: US 2025/0032664 A1
(43) Pub. Date: Jan. 30, 2025

(54) TISSUE DISPLACEMENT FOAM

(71) Applicant: Mayo Foundation for Medical Education and Research, Rochester, MN (US)

(72) Inventor: Jeremy F. McBride, Eau Claire, WI (US)

(21) Appl. No.: 18/841,466

(22) PCT Filed: Mar. 15, 2022
(86) PCT No.: PCT/US2022/020336

§ 371 Date: Aug. 26, 2024

(51) Int. Cl. A61L 24/00; A61L 24/10

(52) U.S. Cl. CPC A61L 24/0036; A61L 24/0015; A61L 24/108; A61L 2300/102; A61L 2300/204; A61L 2300/252; A61L 2300/406; A61L 2300/418; A61L 2300/442; A61L 2430/36

(57) ABSTRACT

Methods and materials are provided herein that can be used to separate healthy organs and/or tissues from organs and/or tissues to be subjected to, for example, heat or cold (e.g., during ablation procedures). Methods and materials also are provided herein that can be used to embolize blood vessels.

### TECHNICAL FIELD

This document relates to methods and materials that can be used to aid in medical procedures. For example, this document relates to methods and materials that can be used to separate healthy organs and/or tissues from organs and/or tissues to be treated with radiation, burning, or freezing (e.g., during ablation procedures). This document also relates to methods and materials that can be used in embolization procedures to occlude blood vessels.

### BACKGROUND

Interventional radiologists are regularly called upon to embolize blood vessels. Embolization often is done at the location of internal bleeding, but the procedure also can be carried out in elective scenarios. Embolization can be performed with the intent of permanent/long lasting occlusion, or temporary materials can be used to achieve temporary occlusion that lasts for about two to four weeks. For permanent embolization, liquid embolics in the form of glue and/or polymerizing compounds can be used. For temporary embolization, gel foam is the only material that is currently used in the United States with any regularity. Temporary embolization can have one or more advantages as compared to permanent embolization. For example, temporary embolization is less likely to cause tissue/organ ischemia, it allows recanalization of the vessels, and there is no permanent imaging artifact to limit visibility on subsequent imaging-unlike coils and some liquid embolics.

Image-guided percutaneous ablation is an effective and safe method to treat tumors in the liver, kidneys, lungs, and musculoskeletal system. Ablation procedures involve using image-guidance provided by real-time ultrasound or CT fluoroscopy to visualize the target tumor and advance needles to the lesion. Appropriate placement of the needles results in thermal destruction of the target tumor with either freezing or heat (e.g., via radiofrequency or microwave energy). While extending the ablation zone just beyond the tumor's edge can be desirable for an adequate margin, there is also a risk of temperature-related injury to adjacent organs and tissues. To protect adjacent tissues, fluid (e.g., normal saline or a similar osmolar-appropriate fluid) or CO₂ can be injected at a tissue plane or the edge of an organ to increase the margin of safety of an ablation zone by physically displacing nontarget organs (e.g., stomach, bowel, pancreas, liver, spleen, and/or kidney) or tissues (e.g., pleura, chest/abdominal wall, muscle) away from the ablation zone. However, since fluid seeks the lowest point in the body and gas seeks the highest point in the body, the accuracy and efficacy of these organ and tissue displacement techniques is limited.

### SUMMARY

This document is based, at least in part, on the development of an albumin-containing foam having a stability in vivo of at least 50% for at least 30 minutes, such that the foam maintains at least half of its original volume for at least 30 minutes after injection into a mammal. The foam provided herein can be used, for example, for organ and/or tissue displacement during ablation procedures, and also can be used in embolization procedures (e.g., as a temporary embolic). The foam provided herein provides an improvement over current techniques used to temporarily displace tissues and organs during ablation procedures. Current techniques typically involve injecting CO₂ or saline through a needle to create a buffer and/or to displace vital structures while using ablation needles to kill nearby tumors with ice or microwave radiation. In some cases, the foam provided herein can be made primarily from serum albumin, yielding a composition that is much more viscous than saline and can stay more localized after injection to provide a more precise and reproducible method of organ/tissue displacement. The foam also is innocuous and can naturally degrade into its absorbable components of protein (e.g., albumin) and CO₂ or ambient air. Further, the foam is viscous and therefore can remain localized at the point of injection, better protecting the desired structures from the ablative energy of the probe but allowing sufficient energy to achieve proper margins in the tumor kill-zone.

This document provides methods and materials that can be used in clinical procedures such as embolization procedures and ablation procedures. In some cases, for example, this document provides compositions in the form of inert fluids that can be injected (e.g., through a small caliber needle) into the body, where the fluids can expand and cause displacement of viscera and/or other anatomic structures, increasing the safety of image-guided interventions. In some cases, this document provides compositions in the form of inert fluids that can be injected into blood vessels, where the fluids can expand and occlude the vessel. In some cases, a composition provided herein can include albumin (e.g., agitated albumin) formulated to result in an inert protein “meringue.” In some cases, other ingredients (e.g., thrombin) can be included. In some cases, CO₂ also can be included in the foam, allowing for visualization under fluoroscopy without the use of iodine-based contrast agents. The viscosity of the foam should allow better control of the injected agent. Methods for using the compositions provided herein to treat mammals in need of a medical procedure (e.g., an embolization procedure or an ablation procedure) also are provided herein.

In general, one aspect of this document features a method for protecting tissue within a mammal. The method can include, or consist essentially of, administering to said mammal a foam composition containing a mammalian albumin polypeptide and a gas, wherein said administering comprises injecting an amount of said foam composition into said mammal at a site between a target tissue and a non-target tissue, such that said foam physically separates said target tissue from said non-target tissue, and subjecting said mammal to an ablation procedure directed to said target tissue, wherein said foam protects said non-target tissue from damage by said ablation procedure. The mammal can be is a human. The albumin polypeptide can be a human albumin polypeptide. The gas can be air or carbon dioxide. The foam can consist essentially of said mammalian albumin polypeptide and said gas. The foam composition can further include an additive. The additive can be protamine or lidocaine. The additive can be a therapeutic agent (e.g., an antibiotic or a coagulant). In some cases, the therapeutic agent can be an antibiotic selected from the group consisting of penicillins, tetracyclines, cephalosporins, quinolones, lincomycins, macrolides, sulfonamides, glycopeptides, aminoglycosides, and carbapenems. In some cases, the therapeutic agent can be a coagulant selected from the group consisting of thrombin, clotting factors of the coagulation cascade, zinc, and antifibrinolytic drugs. The additive can be a contrast agent. The contrast agent can be an iodine-based contrast agent (e.g., iohexol).

In another aspect, this document features a method for embolizing a blood vessel. The method can include, or consist essentially of, injecting into said blood vessel an amount of a foam composition effective to embolize said blood vessel, wherein said foam comprises a mammalian albumin polypeptide and a gas. The mammal can be a human. The albumin polypeptide can be a human albumin polypeptide. The gas can be air or carbon dioxide. The foam can consist essentially of said mammalian albumin polypeptide and said gas. The foam composition can further include an additive. The additive can be protamine or lidocaine. The additive can be a therapeutic agent (e.g., an antibiotic or a coagulant). In some cases, the therapeutic agent can be an antibiotic selected from the group consisting of penicillins, tetracyclines, cephalosporins, quinolones, lincomycins, macrolides, sulfonamides, glycopeptides, aminoglycosides, and carbapenems. In some cases, the therapeutic agent can be a coagulant selected from the group consisting of thrombin, clotting factors of the coagulation cascade, zinc, and antifibrinolytic drugs. The additive can be a contrast agent. The contrast agent can be an iodine-based contrast agent (e.g., iohexol).

In another aspect, this document features a method for inducing thrombosis at a biopsy site in a tissue of a mammal. The method can include, or consist essentially of, obtaining a tissue sample from a selected location in said mammal, and administering to said mammal a foam composition comprising a mammalian albumin polypeptide, thrombin, and a gas, wherein said administering comprises injecting an amount of said foam composition into said mammal at said selected location, wherein said foam induces thrombosis at said selected location. The mammal can be a human. The albumin polypeptide can be a human albumin polypeptide. The gas can be air or carbon dioxide.

In another aspect, this document features a method for inducing thrombosis in a blood vessel of a mammal. The method can include, or consist essentially of, injecting into said blood vessel of said mammal a foam composition comprising a mammalian albumin polypeptide, thrombin, and a gas, wherein said injected foam induces thrombosis in said blood vessel. The mammal can be a human. The albumin polypeptide can be a human albumin polypeptide. The gas can be air or carbon dioxide.

In still another aspect, this document features a foam composition. The foam composition can contain a mammalian albumin polypeptide and a gas, wherein said composition has a stability of at least 50% after 30 minutes in vivo. The albumin polypeptide can be a bovine albumin polypeptide or a human albumin polypeptide. The gas can be air or carbon dioxide. The foam composition can consist essentially of said mammalian albumin polypeptide and said gas. The foam composition can further contain an additive. The additive can be protamine or lidocaine. The additive can be a therapeutic agent (e.g., an antibiotic or a coagulant). In some cases, the therapeutic agent can be an antibiotic selected from the group consisting of penicillins, tetracyclines, cephalosporins, quinolones, lincomycins, macrolides, sulfonamides, glycopeptides, aminoglycosides, and carbapenems. In some cases, the therapeutic agent can be a coagulant selected from the group consisting of thrombin, clotting factors of the coagulation cascade, zinc, and antifibrinolytic drugs. The additive can be a contrast agent. The contrast agent can be an iodine-based contrast agent (e.g., iohexol).

In another aspect, this document features methods for making a foam composition provided herein.

In some cases, the methods provided herein can include foaming with a frothing device (e.g., a whisk), with or without albumin refolding. The methods can include at least some of the steps listed below. It is noted that when refolding is used, steps (c) and (d) can be skipped, and step (b) can be followed by steps (e) to (m). The steps can be as follows:
(a) placing an amount of an albumin (e.g., bovine serum albumin (BSA)) solution into a receptacle;
(b) suspending a frothing device in the receptacle such that the frothing device does not contact an internal surface of the receptacle;
(c) activating the frothing device (e.g., at a high speed) to introduce a gas (e.g., air) into the albumin and generate a foam;
(d) removing the foam from the receptacle and placing it into the barrel of a syringe;
(e) when refolding is used, activating the frother (e.g., at a relatively low speed);
(f) adding an acid (e.g., HCl) drop-wise to the albumin during the first portion of the frothing;
(g) adding a base (e.g., NaOH) drop-wise to the albumin during the last portion of the frothing, such that the pH of the foam approximates the original pH of the albumin solution;
(h) after the frothing is completed, removing the frothing device from the foam and transferring foam from the frothing device to the receptacle;
(i) removing the foam from the beaker and placing it into a first syringe;
(j) connecting the first syringe to a first port of a three-way valve having a piece of tubing connected to a second port and a second syringe connected to a third port, and expelling air from the first syringe via the tubing;
(k) opening the three-way valve to the third port connected to the second syringe, and transferring the foam from the first syringe into the second syringe;
(l) opening the three-way valve to allow gas (e.g., air) to pass into the first syringe through the tubing, where the amount of gas added to the first syringe is such that the total volume of both syringes is a set amount (e.g., 14 mL); and
(m) passing the foam between the first and second syringes for a selected length of time and/or a selected number of times.

In some cases, the methods provided herein can include at least some of the following steps. It is noted that when refolding is used, step (b) can be replaced with steps (c) to (g). The steps can be as follows:
(a) adding an amount of an albumin solution to a syringe, and connecting the syringe to a first port of a three-way valve having a piece of tubing connected to a second port and a second syringe connected to a third port;
(b) passing the albumin solution between the first and second syringes for a selected length of time and/or a selected number of times to generate a foam;
(c) loading the second syringe with an acid (e.g., HCl) and a gas (e.g., air);
(d) passing the acid and gas slowly into the first syringe containing the albumin;
(e) passing the solution between the first and second syringes for a selected length of time and/or a selected number of times;
(f) pushed all of the albumin into one of the syringes, adding a base (e.g., NaOH) to the other syringe, and removing any air from the base-containing syringe; and
(g) passing the base into the albumin-containing syringe, and then passing the mixture between the first and second syringes for a selected length of time and/or a selected number of times to neutralize the acid.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a graph plotting the stability of BSA foams over time, where the foams were prepared by two different methods and with or without refolding of the BSA protein by pH adjustment.

FIG. 2 is a graph plotting the stability of BSA foams over time, where the foams were prepared with or without a buffer, and with various equilibration times for the refolded samples.

FIG. 3A is a graph plotting the stability of human serum albumin (HSA) foams over time, where the foams were prepared using various equilibration times during refolding.

FIG. 3B is pair of graphs plotting the stability (V₁/V₀) of HSA foams at 30 minutes (left panel) and at 60 minutes (right panel) as a function of pH change at 37° C. The horizontal line in each graph indicates the HSA baseline with no pH change. Hashed bars (furthest right, W) indicate whisk preparation with the same pH change conditions as syringe preparation.

FIG. 4 is a graph plotting the stability of BSA foams over time, where the foams were prepared with or without lidocaine.

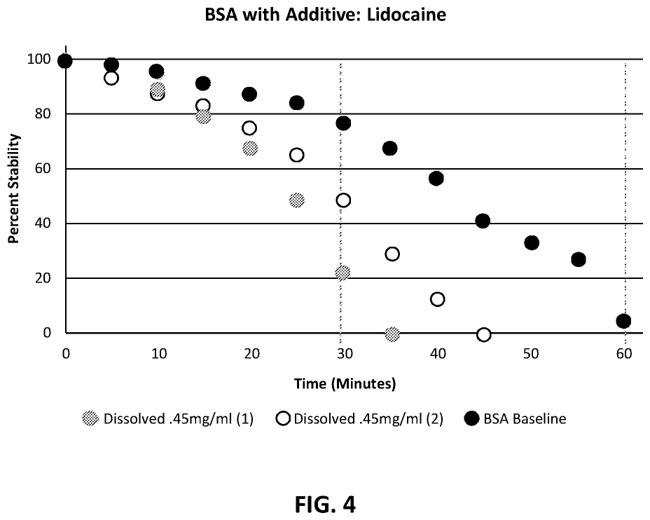

FIG. 5 is a graph plotting the stability of HSA foams over time, where the foams were prepared with refolding at room temperature or at 37° C.

### DETAILED DESCRIPTION

Ablation therapy is a minimally invasive procedure that can be used to destroy abnormal tissue. For example, ablation procedures can be used to destroy (ablate) small amounts of heart tissue in patients with abnormal heart rhythms. Ablation procedures also can be used to destroy tumor cells (e.g., in patients with lung, breast, thyroid, liver, or other cancers). Ablation therapies can be carried out using a probe inserted through the skin, a catheter inserted through a blood vessel, or an energy beam, along with imaging techniques to guide the procedure. Abnormal tissue can be destroyed or impaired using as heat (e.g., radiofrequency ablation), extreme cold (e.g., cryoablation), lasers, or chemicals.

In some cases, cryoablation (also referred to as percutaneous cryoablation, cryosurgery, or cryotherapy) can be used to treat cancer by killing tumor cells with extreme cold. During cryoablation procedures, a cryoprobe is inserted through the skin and directly into a tumor. A gas pumped into the cryoprobe is used to freeze the tissue, which is then allowed to thaw before repeating the freezing and thawing process several times. Cryoablation can be used as a primary treatment for, without limitation, bone cancer, cervical cancer, kidney cancer, liver cancer, lung cancer, and prostate cancer.

Embolization is a treatment in which a catheter is used to inject an embolic substance (e.g., metallic coils, glue, or small particles) into an artery in order to block the flow of blood through the artery. In some cases, for example, embolization can be used to treat excessive or prolonged bleeding, such as bleeding that occurs with trauma to the body, in chronically injured arteries (pseudoanneurusms), in endoleaks after endovascular aneurysm repair (EVAR) of aortic aneurysms, or in vascular malformations, for example. In some cases, embolization can be used to block the blood vessels of tumors or uterine fibroids, thus starving the tumors or fibroids and causing them to shrink and die.

This document provides albumin-based foams that can be used in ablation procedures, embolization procedures, or other medical procedures (e.g., to stop bleeding after a biopsy, such as a lung biopsy, for example). In some cases, the foams provided here can be used to displace and insulate organs and/or tissues during ablation procedures, by separating a tissue, organ, or tumor to be ablated from an adjacent tissue or organ that is not to be ablated. In some cases, the foams provided herein can be used as embolic agents to at least temporarily occlude blood vessels.

In some cases, a foam provided herein can be used in combination with radiation treatment to protect non-target tissue from damage that the radiation otherwise may cause. For example, a foam provided herein can be placed (e.g., by injection) between a target tissue or organ and a non-target tissue or organ, where the target tissue or organ is to be treated with radiation, and where the non-target tissue or organ is adjacent to the target tissue or organ. In some cases, for example, the non-target tissue can be behind or beside the target tissue, in relation to the direction from which the radiation treatment will be administered. The presence of the foam can protect the non-target tissue from the radiation, and can reduce the likelihood that the non-target tissue will be adversely affected by the radiation.

The foams provided herein contain an albumin polypeptide solution in combination with a gas. The foams provided herein can have any or all of the following characteristics and capabilities:
inclusion of biocompatible, FDA approved materials;
a physiological pH of 6.5-7.5 to avoid causing internal harm;
stiffness sufficient to displace tissue;
minimal liquid separation;
the ability to protect tissues and organs from collateral ablation damage;
the ability to provide insulation from the thermal energy of an ablation probe;
the ability to maintain contact with target tissues;
the ability to maintain its mechanical properties during the duration of a medical procedure;
stability during the duration of a medical procedure (e.g., for an hour or more); and
the ability to maintain its mechanical properties at human body temperature (37° C.).

The foams provided herein can contain any appropriate albumin polypeptide. In some cases, for example, a foam provided herein can contain BSA or HSA. In some cases, the albumin polypeptide can be an isolated or substantially pure albumin polypeptide. The term “isolated” as used herein with reference to a polypeptide means that the polypeptide (1) is not associated with proteins found in nature, (2) is free of other proteins from the same source (e.g., free of human proteins), (3) is expressed by a cell from a different species, or (4) does not occur in nature. The term “substantially pure” as used herein with reference to a polypeptide means the polypeptide is substantially free of other polypeptides, lipids, carbohydrates, and nucleic acid with which it is naturally associated. A substantially pure polypeptide can be any polypeptide that is removed from its natural environment and is at least 60 percent pure. A substantially pure polypeptide can be at least about 65, 70, 75, 80, 85, 90, 95, or 99 percent pure, or about 65 to 75, 75 to 80, 80 to 85, 85 to 90, 90 to 95, or 95 to 99 percent pure. Typically, a substantially pure polypeptide will yield a single major band on a non-reducing polyacrylamide gel. In some embodiments, a substantially pure polypeptide can be a chemically synthesized polypeptide.

The foams provided herein can be generated by mixing a gas into an albumin-containing solution, and thus can contain (or consist essentially of, or consist of) an albumin solution and a gas. The foams can contain any appropriate amount of albumin in solution. In some cases, for example, a foam can include a solution that contains about 0.01 g/mL to about 2 g/mL albumin (e.g., about 0.01 to about 0.05 g/mL, about 0.05 to about 0.1 g/mL, about 0.1 to about 0.2 g/mL, about 0.2 to about 0.3 g/mL, about 0.3 to about 0.5 g/mL, about 0.5 to about 0.75 g/mL, about 0.75 to about 1 g/mL, about 1 to about 2 g/mL, about 0.1 g/mL, about 0.25 g/mL, or about 0.5 g/mL albumin). In some cases, an albumin solution can contain about 2 wt % to about 50 wt % albumin (e.g., about 2 to about 5 wt %, about 5 to about 10 wt %, about 10 to about 20 wt %, about 20 to about 30 wt %, about 30 to about 50 wt %, about 10 wt %, about 20 wt %, about 25 wt %, or about 30 wt % albumin). Suitable solvents include, without limitation, water, saline, phosphate buffered saline (PBS), and dextrose 5% in water (D5W). In some cases, a solvent can contain one or more stabilizing additives such as, without limitation, sodium acetyltryptophanate and/or sodium caprylate.

The foams provided herein also can contain any appropriate type of gas. For example, a foam provided herein can contain ambient air, CO₂, O₂, or any combination thereof (e.g., a combination of O₂, CO₂, and ambient air). When included in a foam, CO₂ can allow for visualization under fluoroscopy without the use of iodine-based contrast agents. In addition, CO₂ can be rapidly absorbed without clinical consequence.

The albumin and gas in a foam provided herein can be present in any ratio. In some cases, for example, a foam provided herein can contain albumin and gas at an albumin:gas ratio of about 1:1 to about 1:5 (e.g., about 1:1, about 1:2, about 1:3, about 1:4, about 1:5, about 1:1 to about 1:3, about 1:2 to about 1:4, or about 1:3 to about 1:5). In some cases, a foam provided herein can have an albumin:gas ratio of about 1:3.

In some cases, a foam provided herein can include one or more ingredients in addition to albumin and gas. For example, a foam provided herein can include one or more polypeptides in addition to the albumin, one or more therapeutic agents, and/or one or more contrast agents (e.g., to facilitate visualization).

For example, in some cases, an albumin-based foam provided herein can contain thrombin, a blood protein that causes clot formation by converting fibrinogen to fibrin. Foams containing thrombin can be useful as, without limitation, temporary liquid or semi-liquid embolics that cause thrombosis when they come into contact with blood. Such embolic foams can provide a liquid-like temporary embolic agent that can be used in, for example, interventional radiology procedures or to induce clotting as a treatment for, e.g., post-biopsy bleeding. Any appropriate amount of thrombin can be included in a foam provided herein. For example, a foam can be generated from a solution that includes about 10% to about 50% by volume (e.g., about 10% to about 20%, about 15% to about 25%, about 20% to about 30%, about 30% to about 40%, or about 40% to about 50%, by volume) of a thrombin solution (e.g., a solution containing about 2500 units of thrombin/mL). In some cases, a solution from which a foam is generated does not contain more than 10% by volume of a thrombin solution. Thrombin-containing foams have unique properties that can provide advantages over existing embolic technologies. These advantages can include, for example, the semi-solid nature of the foam that allows it to conform to vessels and even flow into smaller vessels; its potential to provide rapid thrombosis with more control and visualization than injected thrombin without a foam carrier; the absence of permanent artifact on subsequent imaging (which may result with the use of metal coils and liquid embolics); and the potential for vessel recanalization given its composition of blood products and CO₂. This last characteristic can make the foam less likely to cause serious complications in non-target embolization and may allow “temporary” embolization, which can be desirable in clinical circumstances. While there are embolic agents that have these characteristics, none have the unique combination provided by the thrombotic foam provided herein.

In some cases, an albumin-based foam provided herein can contain lidocaine (e.g., lidocaine HCl), a local anesthetic that is often used to prevent pain by blocking the signals at the nerve endings in the skin. Without being bound by a particular mechanism, the inclusion of lidocaine in a foam provided herein may increase the volume of foam that is produced, possibly due to a decrease in surface tension. The addition of lidocaine also can, in some cases, increase the stability of the foam. Any appropriate amount of lidocaine can be included in a foam provided herein. For example, a foam can be generated from a solution that includes about 0.5% to about 5% by volume (e.g., about 0.5% to about 1%, about 1% to about 2%, about 2% to about 3%, about 3% to about 4%, or about 4% to about 5%, by volume) of lidocaine (e.g., a 1% lidocaine solution). In some cases, a solution from which a foam is generated does not contain more than 10% by volume of a lidocaine solution.

In some cases, an albumin-based foam provided herein can contain protamine, a specific antagonist that can neutralize heparin-induced anticoagulation. Without being bound by a particular mechanism, protamine can improve the foaming behavior of an albumin solution, possibly as a result of its electrostatic interactions with albumin. Protamine is known to be a strong cation (Glaser et al., Food Hydrocolloids 21:495-506, 2007). Any appropriate amount of protamine can be included in a foam provided herein. For example, a foam can be generated from a solution that includes about 3% to about 50% by volume (e.g., about 3% to about 5%, about 5% to about 10%, about 10% to about 20%, about 20% to about 30%, about 30% to about 40%, or about 40% to about 50%, by volume) of a protamine solution (e.g., a 10 mg/mL protamine solution). In some cases, a solution from which a foam is generated does not contain more than 10% by volume of a protamine solution.

In some cases, an albumin-based foam can contain one or more therapeutic agents. Any appropriate therapeutic agent can be included in a foam provided herein. Suitable types therapeutic agents include, for example, antibiotics, coagulants, and combinations thereof. Examples of antibiotics that can be included in a foam provided herein include, without limitation, penicillins, tetracyclines, cephalosporins, quinolones, lincomycins, macrolides, sulfonamides, glycopeptides, aminoglycosides, and carbapenems. Examples of coagulants that can be included in a foam provided herein include, without limitation, thrombin, clotting factors from the coagulation cascade (e.g., fibrinogen/Factor I, prothrombin/Factor II, tissue thromboplastin/Factor III, Factor VII, Factor VIII, Factor IX, Factor X, Factor XI, Factor XII, or Factor XIII), zinc, and antifibrinolytic drugs (e.g., aprotinin, tranexamic acid (TXA), epsilon-aminocaproic acid, and aminomethylbenzoic acid).

Any appropriate amount of a therapeutic agent can be included in a foam provided herein. For example, a foam can be generated from a solution that includes about 0.5% to about 50% by volume (e.g., about 0.5% to about 2.5%, about 2.5% to about 5%, about 5% to about 10%, about 10% to about 20%, about 20% to about 30%, about 30% to about 40%, or about 40% to about 50%, by volume) of a solution containing a therapeutic agent (e.g., a 1% to 50% solution of the therapeutic agent). In some cases, a solution from which a foam is generated does not contain more than 10% by volume of a solution containing a therapeutic agent.

In some cases, an albumin-based foam can include an iodinated contrast agent. For example, a foam can include iohexol (OMNIPAQUE™), an iodinated contrast agent used during CT-guided procedures. The iohexol can, in some cases, enhance the visibility of the foam. The inclusion of iohexol in a foam provided herein also can facilitate determination of what happens to the albumin solution within the tissue after the foam breaks down. In some cases, a foam also can contain gadolinium, which can provide MRI compatibility. Any appropriate amount of an iodinated contrast agent or gadolinium can be included in a foam provided herein. For example, a foam can be generated from a solution that includes about 1% to about 7% by volume (e.g., about 1% to about 2%, about 2% to about 3%, about 3% to about 4%, about 4% to about 5%, about 5% to about 6%, or about 6% to about 7% by volume) of a solution containing an iodinated contrast agent and/or gadolinium (e.g., a 0.1% to 50% solution of the contrast agent and/or the gadolinium). In some cases, a solution from which a foam is generated does not contain more than 10% by volume of a solution containing an iodinated contrast agent and/or gadolinium.

Other components that can be included in an albumin-based foam provided herein include, for example, one or more polysaccharides. In some cases, cationic polysaccharides can be included, as such molecules may stabilize exposed charged amino acids of denatured albumin protein (Miquelim et al., Food Hydrocolloids 24:398-405, 2010). Any appropriate amount of a polysaccharide can be included in a foam provided herein. For example, a foam can be generated from a solution that includes about 3% to about 20% by volume (e.g., about 3% to about 5%, about 5% to about 10%, about 10% to about 15%, or about 15% to about 20%, by volume) of a solution containing an one or more polysaccharides (e.g., a 0.1% to 50% solution of the one or more polysaccharides). In some cases, a solution from which a foam is generated does not contain more than 10% by volume of a polysaccharide solution.

This document also provides methods for making the foams described herein. In general, the methods provided herein for making albumin-based foams include making or obtaining an albumin solution and combining the solution with gas (e.g., air or CO₂) to produce a foam. In some cases, an albumin solution can be obtained commercially, such as when the albumin is in a commercially available pharmaceutical grade HSA solution. In some cases, an albumin solution can be prepared by dissolving an albumin powder in a suitable fluid carrier (e.g., sterile water, saline, or DSW). The albumin in the solution can have any appropriate concentration. For example, an albumin solution used to generate a foam can contain albumin at about 0.01 g/mL to about 2 g/mL (e.g., about 0.01 to about 0.05 g/mL, about 0.05 to about 0.1 g/mL, about 0.1 to about 0.2 g/mL, about 0.2 to about 0.3 g/mL, about 0.3 to about 0.5 g/mL, about 0.5 to about 0.75 g/mL, about 0.75 to about 1 g/mL, about 1 to about 2 g/mL, about 0.1 g/mL, about 0.25 g/mL, or about 0.5 g/mL). In some cases, an albumin solution used to generate a foam can contain albumin at about 0.25 g/mL. When generating an albumin solution from powdered albumin, the powdered albumin can be added to the fluid at about 2 wt % to about 50 wt % (e.g., about 2 to about 5 wt %, about 5 to about 10 wt %, about 10 to about 20 wt %, about 20 to about 30 wt %, about 30 to about 50 wt %, about 10 wt %, about 20 wt %, about 25 wt %, or about 30 wt %).

When an ingredient in addition to the albumin (e.g., thrombin, lidocaine, protamine, a therapeutic agent, or another additive) is to be included in a foam provided herein, the further ingredient can be added to the albumin solution (e.g., when the albumin is dissolved or after the albumin is dissolved) at any appropriate concentration.

After a solution containing albumin with or without one or more additives is obtained or generated, a foam can be prepared using any appropriate method.

In some cases, an albumin-based solution can be combined with a gas (e.g., air or CO₂) and passed back and forth between two syringes to generate a foam. For example, the syringe bodies can be in fluid communication with each other via a tube or a valve (e.g., a three-way valve) connected to the luer of each syringe. At the beginning of the foam preparation process, an albumin-containing solution can be placed into the body of one syringe, and gas (e.g., air or CO₂) can be loaded into the second syringe. The gas can be injected into the solution through the tube or valve connecting the two syringes, and the mixture can then be passed back and forth until a foam is achieved.

Any appropriate number of passes between the syringes over any appropriate length of time can be carried out to achieve a foam. For example, the combined albumin solution and gas can be passed between the syringes about 20 to 500 times (e.g., about 25 to 50 times, about 50 to 100 times, about 100 to 200 times, about 200 to 300 times, about 300 to 400 times, or about 400 to 500 times). The passing back and forth between the syringes can take place for at least about 20 seconds (e.g., from about 20 to about 30 seconds, from about 30 to about 45 seconds, from about 45 to about 60 seconds, from about 60 to about 90 seconds, from about 90 to about 120 seconds, or from about 120 to about 180 seconds).

In some cases, an airstone or inline filter can be included in the syringe system to modulate the size of bubbles in the foam generated therein. The number of times the foam passes through a filter can affect the bubble size of the foam, which in turn can affect the volume of the foam produced, as well as the stability of the foam. In some cases, a gas can be injected through an airstone into an albumin solution in a single pass. In addition to modulating the overall size of the bubbles, the use of an airstone can lead to generation of bubbles having a fairly uniform size.

In some cases, a foam can be prepared using a whisk, a blender (e.g., an immersion blender), a frother (e.g., a milk frother), or a combination thereof. An albumin solution can be placed in a receptacle having an appropriate size (e.g., a beaker with a volume of 20 mL, 50 mL, or 100 mL), for example, and a whisk, blender, or frother can be suspended in solution. The solution can then be mixed for any appropriate length of time, at any appropriate speed. In some cases, for example, the solution can be mixed for about 30 seconds to about 10 minutes (e.g., about 30 to about 60 seconds, about 60 seconds to about 90 seconds, about 1 to about 2 minutes, about 2 to about 3 minutes, about 3 to about 4 minutes, about 4 to about 5 minutes, about 5 to 7 minutes, or about 7 to 10 minutes). The solution and foam being generated can be mixed on a low speed setting, a medium speed setting, a high speed setting, or a combination thereof. For example, the solution and foam being generated can be mixed for a first period of time at a first speed, a second period of time at a second speed, and so forth. In some cases, after mixing, the foam can be removed from the receptacle and evaluated (e.g., for stability) or used to treat a mammal.

In some cases, the pH of the solution and foam being generated can be modulated during mixing to allow the albumin to unfold and refold. For example, the pH can be reduced during foam preparation by adding a suitable amount of an acid (e.g., HCl) to the frothing albumin solution. The acid can be added to gradually reduce the pH of the mixture. After the pH has been reduced to an appropriate level (e.g., to a pH of about 0.5 to about 4, about 1 to about 3, or about 1.5 to about 2.5), a base (e.g., NaOH) can be added to the frothing albumin solution. The base can be added to gradually increase the pH to an appropriate level (e.g., to a pH of about 6.5 to about 7.5, about 6.8 to about 7.3, or about 7). The foam can then be removed from the receptacle, and can be evaluated (e.g., for stability) or used to treat a mammal.

In some cases, a foam can be prepared using a combination of the methods described herein. For example, an albumin solution can be mixed with a whisk or a frother to generate a foam, and the foam can be placed into a two-syringe system and passed back and forth as described above.

The physical properties and characteristics (e.g., stability, volume, firmness, and surface tension) of a foam provided herein can be adjusted by, for example, modulating the pH and/or the temperature of the foam during preparation. For example, pH can influence the stability and surface tension of albumin foams. In some cases, pH modulation (e.g., reducing the pH and then increasing the pH back to a physiological level) can be used to cause unfolding and refolding of albumin proteins. Including unfolding and refolding in a foam preparation method can affect the properties of the resulting foam, such as the stability of the foam and its resistance of the foam to external forces (Mleko et al., LWT 40:908-914, 2006). The pH of a foam can be measured using, for example, a pH meter. The initial pH of an albumin-containing solution used in the foam preparation methods provided herein can be, for example, from about 4.5 to about 6.0 (e.g., about 4.5 to about 4.8, about 4.8 to about 5.0, about 5.0 to about 5.3, about 5.3 to about 5.5, about 5.5 to about 5.8, or about 5.8 to about 6.0). When the pH is modulated during foam preparation to promote unfolding and refolding of the albumin, the pH can be reduced to a value of about 0.5 to about 4, and then increased to about 6.5 to about 7.5.

The foams provided herein typically have a stability in vivo of at least 50% (e.g., at least 55%, at least 60%, at least 65%, at least 70%, at least 75%, at least 80%, at least 85%, at least 90%, or at least 95%) for at least 30 minutes (e.g., at least 45 minutes, at least 60 minutes, at least 75 minutes, or at least 90 minutes), such that the foam maintains at least half of its original volume/thickness for at least 30 minutes after injection into a mammal. The volume and/or thickness of a foam in vivo can be assessed by CT scanning, for example.

The volume of a foam produced by the methods provided herein can be measured based, for example, on the markings on the syringe(s) in which the foam is placed after it is prepared. In some cases, a foam can be placed into a graduated cylinder for in vitro evaluation of its volume and/or stability. For example, the stability of a foam can be evaluated by placing it into a graduated cylinder (e.g., by injecting it out of the syringe(s) in which it was generated), and incubating the graduated cylinder at 37° C. (e.g., in a water bath). The volume of the foam can be monitored over time, and any decrease in the volume of the foam as a function of time can be determined. In some cases, a weight (e.g., a 5 g, 10 g, or 20 g weight) can be placed on top of the foam in the graduated cylinder, and the volume of the foam can be monitored over time.

The percent stability of a foam can be calculated using the following formula:

$$Percent stability = 100 \times \frac{( collapse height - liquid separation )}{initial height of foam}$$

In some cases, other methods can be used to characterize the foams provided herein. These can include, for example, rheology and/or pendant drop tensiometry (see, e.g., Miquelim et al., Food Hydrocolloids, 24(4):398-405, 2010; Mleko et al., LWT, 40:908-914, 2007; and Glaser et al., Food Hydrocolloids, 21:495-506, 2007). A device containing animal tissue (e.g., bovine or porcine liver tissue, as described in Example 9 herein) also can be used to characterize foams in contact with tissue. For example, such a device can be used to assess foam stability and stiffness in contact with biologic tissue rather than the glass used in more standard tests.

This document also provides methods for using the foams provided herein. In general, the methods can include injecting a suitable amount of an albumin-containing foam provided herein into an appropriate location within a mammal. In some cases, for example about 10 mL to about 1000 mL (e.g., about 10 mL to about 25 mL, about 25 mL to about 50 mL, about 50 mL to about 100 mL, about 100 mL to about 250 mL, about 250 mL to about 500 mL, about 500 mL to about 750 mL, or about 750 mL to about 1000 mL) of a foam provided herein can be injected into a mammal to displace a tissue or tumor to be ablated from adjacent tissue(s) or organ(s) that are not to be ablated. The volume of foam injected can be selected to achieve any suitable displacement distance between adjacent tissues or organs. For example, an amount of foam can be injected to achieve displacement between adjacent tissues or organs of about 0.25 cm to about 3 cm (e.g., about 0.25 cm to about 0.5 cm, about 0.5 cm to about 1 cm, about 1 cm to about 1.5 cm, about 1.5 cm to about 2 cm, or about 2 cm to about 3 cm), which can provide adequate ablation margins while maintaining a safe separation from adjacent tissues/organs that are not to be ablated. A foam can be injected into a mammal using, for example, a syringe (e.g., a syringe connected to a needle having a size from about 25 gauge to about 18 gauge), a catheter, an introducer, and/or a dilator.

Any appropriate mammal can be treated as described herein. For example, humans, non-human primates, dogs, cats, horses, cows, pigs, sheep, mice, rabbits, and rats can be injected with a foam provided herein. In some cases, the mammal can be a human patient slated to undergo an ablation procedure, and the foam can be injected such that it is positioned between a tissue or tumor to be ablated and one or more tissues or organs adjacent to the tissue or tumor.

When ablating tumor tissue near a liver of a human, for example, between about 10 mL and about 1000 mL of a foam described herein can be injected into a human between the liver and the tumor tissue, to move the liver from about 0.5 cm to about 2 cm away from the tumor. Once injected, the foam provided herein typically is sufficiently set up such that an ablation procedure can be carried out right away. When treating a mammal, the amount of foam injected can vary depending on the specific location of the tumor and the tissue(s)/organ(s)/structure(s) that the foam is intended to protect, and the characteristics of the space into which the foam is to be injected (e.g., the natural tissue planes, the mobility and/or size of the organ(s), potential heat sinks, sensitivity of the tissue/organ, etc.).

In addition to use during ablation and embolization procedures, a foam provided herein can be injected into a mammal for other applications. For example, a foam containing an embedded antibiotic can be injected at an injury site, such that the foam would dissipate with time and the antibiotic would be left in place. As another example, a foam containing thrombin can be injected at a site with tissue damage. Thrombin can also be included in a foam used during an ablation procedure when the ablation probe is being removed from the patient, to promote coagulation and healing of the insertion site.

The invention will be further described in the following examples, which do not limit the scope of the invention described in the claims.

### EXAMPLES

### Example 1—Albumin Compositions and Foam Preparation

Foams with varying stability were prepared using ovalbumin powder, bovine serum albumin (BSA) powder, or pharmaceutical grade human serum albumin (HSA, 0.25 g/mL). For the powdered albumin samples, 25 wt % solutions were prepared with buffer containing sodium octanoate and N-acetyl-tryptophan, or with sterile water.

Two main foam preparation methods were used. In the first method, the solution was passed between two 20 mL syringes, one containing the albumin solution and the other containing air or CO₂ gas, multiple times. In the second method, an immersion blender with a milk frother attachment was used, with controlled depth in solution and time for blending. Foam stability as a function of time and foam stiffness as a function of applied mass were measured in triplicate to compare albumin sources. Briefly, the foams were mixed and placed in a gradated cylinder, and a weight was placed on top of the foam. The volume of foam to liquid was then measured periodically at physiologic temperature. The syringe method gave more consistent results once the procedure was optimized. Both methods are described in more detail as follows.

Albumin Refolding and Foaming Procedure with Milk Frother (Whisk)

1. Using a pipette, 4 mL of BSA were added to a 20 ml beaker (when refolding) or a 50 ml beaker (when not refolding). The 4 mL volume was used because foaming can produce up to 20 mL of volume, and it was observed that 5 mL of albumin could overflow the beaker. When not refolding, even more foam was produced, and a 50 ml beaker was needed to keep overflow from happening. A 20 mL beaker was used when refolding, because foaming did not require as much air and did not create as much volume as a non-refolded foam.

2. A milk frother was suspended in the 20 or 50 mL beaker so that neither the sides nor the bottom of the beaker directly touch the frother at any time during the frothing process. The frother was positioned 1 mm above the bottom of the beaker to prevent the frother from bending and chipping the bottom of the beaker over time. The frother used was the Mueller hand blender, chosen for its ability to add air more effectively than a whisk. A very small magnetic stir bar was be inserted into the center of the frother to prevent the foam from becoming stuck on the inside of the frother. The frother and the beaker were suspended and clamped to a stand and set to rest on top of a container or hot plate that was not turned on. Further, the power button of the frother was taped into the “on” position so that the frother could be turned on by plugging it in, thus alleviating the need to hold the “on” button down for minutes on end.

3. When refolding was not used, frothing was carried out as desired. Various combinations of frother speeds and frothing times were used, depending on the desired outcome. For example, during baseline trials, the frother was kept on high for three minutes and on turbo for three minutes.

4 The foam was scooped out of the beaker with a spatula and quickly inserted into the back of a plastic syringe. To avoid adding air bubbles during this process, foam was only added to the back of the syringe, with little to no air present between the plunger and the start of the foam when the plunger was inserted into the syringe.

When refolding was used, steps 3 and 4 were skipped. When refolding was not used, steps 3 and 4 were followed by steps 14-16, and steps 5-13 were skipped.

5. When refolding was used, the frother was kept on low for six minutes. Keeping the frother at a low speed allowed more time for the HCl and NaOH to spread out before too much foaming happened, preventing the BSA from gelling up, and preventing the mixer from overheating.

6. During the first 3 minutes of frothing, 6M HCl was added drop-wise to the frothing BSA, with one drop added every few seconds.

7. During the last 3 minutes of frothing, 0.6 ml of 4M NaOH was added to the frothing BSA, with one drop being added every 5 seconds. The amounts of acid and base were added to neutralize, so that the final pH of the BSA was as close to the original pH of the BSA as possible. The final product had a consistency similar to that of marshmallow fluff.

8. When frothing was completed, a spatula was used to clean off the frother after it was removed from the foam. The spatula was not inserted between the grooves of the frother, but was quickly run along the outsides and top and bottom of the frother to remove the majority of the foam on the frother. The extra foam was put into the beaker. This step was standardized for reliable results.

9. Next, the foam was scooped out of the beaker with a spatula and quickly inserted into the back of a plastic syringe. To avoid adding air bubbles during this process, foam was added only to the back of the syringe such that there was little to no air between the plunger and the start of the foam when the plunger was inserted into the syringe.

10. The syringe was connected to a three-way valve with a clear plastic tube coming out of the valve parallel to and in line with the syringe body. The valve was open to the tube section and closed to the perpendicular valve opening. All air was expelled from the syringe through the tube connected to the valve opening, and the valve was then opened to the perpendicular tube section.

11. The perpendicular tube section was attached to a second syringe having its plunger pushed all the way down so there was no air inside it. The valve to the second syringe was opened and the foam was pushed into the second syringe, so that there was no foam left in the original syringe.

12. The valve was opened to allow air to pass into the original syringe through the tube. Air was added to the syringe so the total volume of both syringes was 14 mL. For example, if the amount of foam was 7 mL, that meant that 3 mL of air had been added to the foam and only 7 mL of additional air should be added to the second syringe. The original syringe was then closed to the tube and opened to the perpendicular syringe with the foam inside.

13. At this point, one syringe contained 100% foam and the other contained 100% air. A timer was set for 45 seconds, and the foam was passed between the two syringes 90 times. The foam became increasingly dense and strong as it was passed between the syringes. At the end of this step, the BSA had transitioned from a gel-like marshmallow foam substance to a more insulation foam-like substance.

14. Using a long tweezers, the tube was inserted into a 25 mL graduated cylinder. The form was pushed out of the syringe and into the graduated cylinder through the tube. To avoid introducing air bubbles into the foam as it was expelled from the syringe, the tube was held in the center of the graduated cylinder and slowly raised as the foam built up inside the cylinder, keeping the end of the tube slightly submerged in the foam.

15. The desired amount of weight was placed on top of the foam structure, and the initial amount of foam in the beaker was recorded. If the experiment involved a 3D printed weight, it was lowered into the graduated cylinder using a pair of tweezers so it did not impact the foam from falling. The initial amount of foam was equal to the milliliter line at the top of the weight minus the size of the weight in milliliters. For the 10 g weight, this meant that 3.95 mm was subtracted from the top of the weight to get the foam's initial volume.

16. A time lapse video or series of photos was recorded for one hour (e.g., on a phone). This allowed better tracking and calculation of stability over time. Percent stability was calculated as:

$$Percent stability = 100 \times \frac{( collapse height - liquid separation )}{initial height of foam}$$

17. After the time lapse was complete, the foam was allowed to sit until enough liquid was available to be pH tested.

### Albumin Refolding and Foaming Procedure in Syringe

1. A pipette was used to add 4 mL of albumin to a 20 ml syringe. The end of the syringe was connected to a three-way valve with clear plastic tube coming out of the valve parallel and in line with the syringe. The perpendicular section of the three-way valve was connected to a second syringe.

2. A timer was set for 90 seconds, and the solution was passed between the syringes 180 times.

3. Using a long tweezers, the tube was inserted into a 25 mL graduated cylinder, and the foam was passed from the syringe into the cylinder. To avoid introducing air bubbles into the foam structure as it is was expelled from the syringe the tube was held in the center of the graduated cylinder and raised slowly as the foam built up inside the cylinder, keeping the end of the tube slightly submerged in the foam.

4. The desired amount of weight was placed on top of the foam structure and the initial amount of foam in the cylinder was recorded. If a 3D printed weight was used, it was lowered into the graduated cylinder using a pair of tweezers so it did not impact the foam. The initial amount of foam was equal to the mm line at the top of the weight minus the height of the weight in cm. For example, for a 10 g weight, 3.95 mm was subtracted from the top of the weight to arrive at the foam's initial volume. Percent stability was calculated as described above.

5. An hour-long time lapse series of photos or video was recorded (e.g., using a phone) to facilitate tracking and calculation of stability over time. The entire process of adding foam to the graduated cylinder and adding the weight on top took about 1 minute.

6. After the time lapse was complete, the foam was allowed to sit until enough liquid was available to be pH tested.

When refolding was used, step 2 was replaced with steps 7 through 11, followed by steps 3-6. When refolding was not used, only steps 1 through 6 were used.

7. The second syringe was loaded with 0.40 mL of 6M HCl and 10 mL of air.

8. The HCl and air were passed slowly into the syringe containing the albumin.

9. A timer was set for 3 minutes, and the solution was passed between the syringes 60 times. It was noted that although 90 passes would ideally have been completed in 45 seconds to match the baseline procedure, this was not possible with the current procedure due to the high viscosity of the HCl and albumin mixture. Passing the mixture between syringes became almost impossible after 60 passes, depending on the albumin used.

10. The albumin was pushed to one side of the syringe, and 0.6 mL of 4M NaOH was added to the other side of the syringe. During this time the albumin HCl solution was allowed to equilibrate for 3 minutes, taking care to not allow any NaOH to pass to the other side of the syringe during equilibration. The amounts of acid and base were added to neutralize the solution so that the final pH of the solution was as close to the original pH of the albumin as possible. After adding the NaOH, all air was removed from the syringe so that only NaOH remained, ensuring that a minimal amount of additional air was added to the original 10 ml of air. Passing the solution between the syringes during refolding facilitated mixing of the HCl, albumin, and NaOH, because forcing the molecules through the narrow passage between the syringes ensured that the molecules interacted with each other. This was in contrast to the whisk/milk frother method, where the gel-like albumin and HCl mixture could avoid mixing with the NaOH and therefore did not neutralize at a pH close to the original pH of the albumin.

11. The NaOH was passed between the syringes 90 times in 2 minutes to neutralize the HCl.

### Example 2—Data Collection Standards

An updated measurement method following readily available characterization for foams was used to assess stability. Information described elsewhere relating to the field of albumin and foams was used as a new guideline for industry standards in albumin foam data collection, including models for in-lab quantitative measurements of stability through foam collapse. For example, a cost-effective option is to monitor foam collapse in a graduated cylinder (see, e.g., Burapatana et al., Biotechnology for Fuels and Chemicals, 905-911, 2003; Miquelim et al., Food Hydrocolloids, 24(4): 398-405, 2010; and Pycarelle et al., Food Hydrocolloids, 101:105548, 2020). Stability is defined as the percent of foam remaining at a given time. The solution separated at the bottom and was subtracted from the total volume of the foam. Measurements were made more precise by using millimeter marks on the side of the container instead of the volumetric ones etched into the container. Further, using a camera to monitor the foam allowed for constant data collection and the ability to trace foam decay at any given point. These data collection methods, when combined, replicated the abilities of the Krüss Scientific Dynamic Foam Analyzer (DFA 100), which is considered a reliable machine for published data on albumin foams (Richert et al., Journal of Physical Chemistry B, 122(45): 10377-10383, 2018).

### Example 3—Effects of Foaming Method and pH Adjustment on BSA Foam Stability

Because of the cost of HSA, different routes were explored in an attempt to forecast the results for HSA experiments with less expensive and more lab-ready materials (available to ship and store). In addition to its lower cost and ready availability, BSA was used because its protein structure closely resembles that of human albumin, and a BSA solution with buffer compounds can be prepared.

The foam stability experiments typically consisted of generating a foam and them monitoring the volumes of foam and denatured solution in a graduated cylinder at physiologic temperature. To evaluate stiffness, a weight was placed on top of the foam, and the effect on foam volume was assessed. In general, to generate a foam, about 1 mL of HCl to was added to about 8 mL of 25% albumin in a 20 mL syringe, the mixture was agitated with about 20 mL of gas (room air) in a second syringe by passing the mixture back and forth between the syringes via a 3-way stop cock for about 2 minutes. After allowing the foam to set for a 1-2 minutes, about 1 mL of NaOH was added, and the mixture was further agitated between the syringes via the stopcock for about 2 minutes.

The syringe mixing procedure was adjusted to provide better mixing. Initial results showed that insufficient mixing upon addition of acid resulted in gelation, in contrast with a procedure in which the albumin solution was maintained at decreased pH. Optimizing the mixing resulted in increased foam stability when the pH was increased back to physiologic range. A relatively large change in pH (from about 3.5 to about 8) was required in order to obtain the greatest foam stability using these conditions. Specifically, the pH was first adjusted to about 0.6 to achieve protein binding and maximum stability, which occurs at and under a pH of 1 (based on food science work showing increased stability; Liang and Kristinsson, J. Food. Sci., 2005, 70(3):C222-C230). After equilibration, the pH was adjusted back to physiologic pH. Using this procedure, the albumin solution was consistently increased to a pH between 6.5 and 7.5. Foam column collapse was recorded through time-lapse video to track decay.

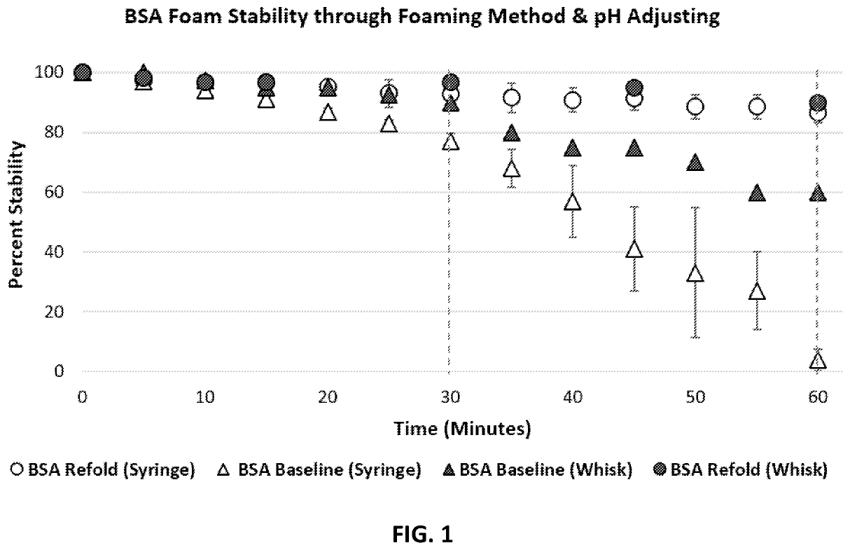

As compared to ovalbumin, BSA more accurately predicted HSA foam stability and stiffness results. Experiments with BSA as a baseline (only BSA and water at 25 wt %, with no buffer and no adjustment to pH) were conducted using the syringe method and the whisk/milk frother method. The results were compared to BSA with refolding (BSA and water at 25 wt %, without buffer but with the adjustments to pH described above). The pH-refolded BSA solutions resulted in more stable foams as a function of time in comparison to the baseline BSA solutions (TABLE 1 and FIG. 1). The same trends were observed with both types of mixing, suggesting that the chemical changes with changing pH are more significant than mechanical changes due to mixing type.

TABLE 1
BSA foam percent stability

| | BSA | BSA | BSA | BSA |
|---|---|---|---|---|
| | Refold | Baseline | Refold | Baseline |
| | (syringe) | (syringe) | (whisk) | (whisk) |
| 30 minute % stability | 93 | 77 | 97 | 90 |
| 60 minute % stability | 87 | 4 | 90 | 60 |

### Example 4—Buffer Effect on BSA Foam Stability

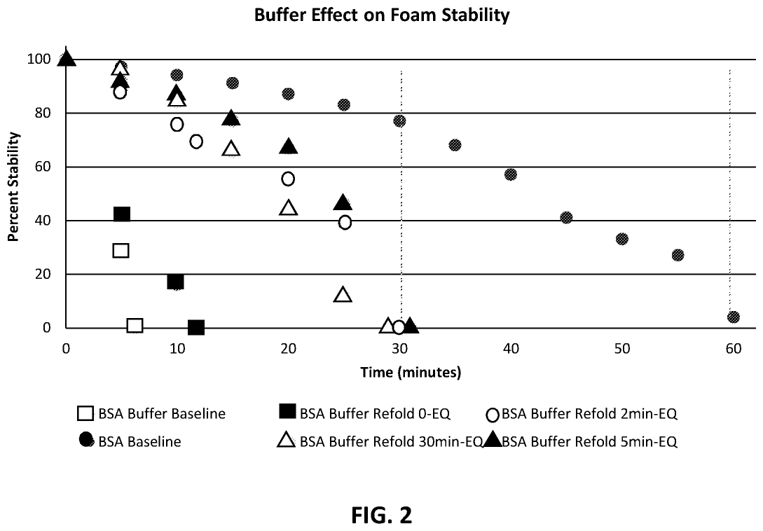

BSA was prepared in a 0.02 M buffer solution of sodium octanoate and N-acetyl-tryptophan (the major components listed for the HSA buffer) to more accurately mimic the HSA buffer solutions. The syringe method was used to mix the acid into the albumin solution, as well as to mix the base into the mixture and create the foam, providing a more homogeneous mixture and more effective change in pH to improve foam stability. Tests were then conducted to determine the effect of the buffer on foam stability. Equilibration time at low pH was also was tested to determine the effect on foam stability. Addition of the buffer decreased the stability of the BSA foam (TABLE 2 and FIG. 2). This was consistent with the difference that was observed between the initial ovalbumin experiments (samples with no buffer were much more stable) and HSA (samples with buffer were less stable over time). These studies also demonstrated that the equilibration time at low pH was a critical variable, and that a minimum equilibration time was needed to maximize foam stability. The BSA foam result trends were consistent with the HSA foam result trends (see Example 5).

TABLE 2
Buffer effect on BSA foam stability

| | | BSA | | | | |
|---|---|---|---|---|---|---|
| | BSA | Buffer | BSA Buffer | BSA Buffer | BSA Buffer | BSA Buffer |
| | Baseline | Baseline | Refold 0-EQ | Refold 2-EQ | Refold 5-EQ | Refold 30-EQ |
| Time of | ~60 min | ~6:20 | ~10:45 | ~30 min | ~31 min | ~29 min |
| total | | | | | | |
| collapse | | | | | | |

### Example 5—HSA Refold Stability and Equilibration Time

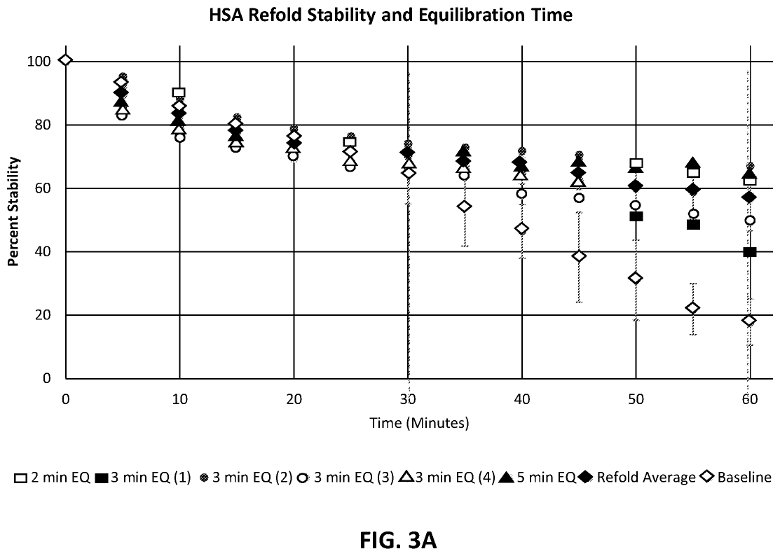

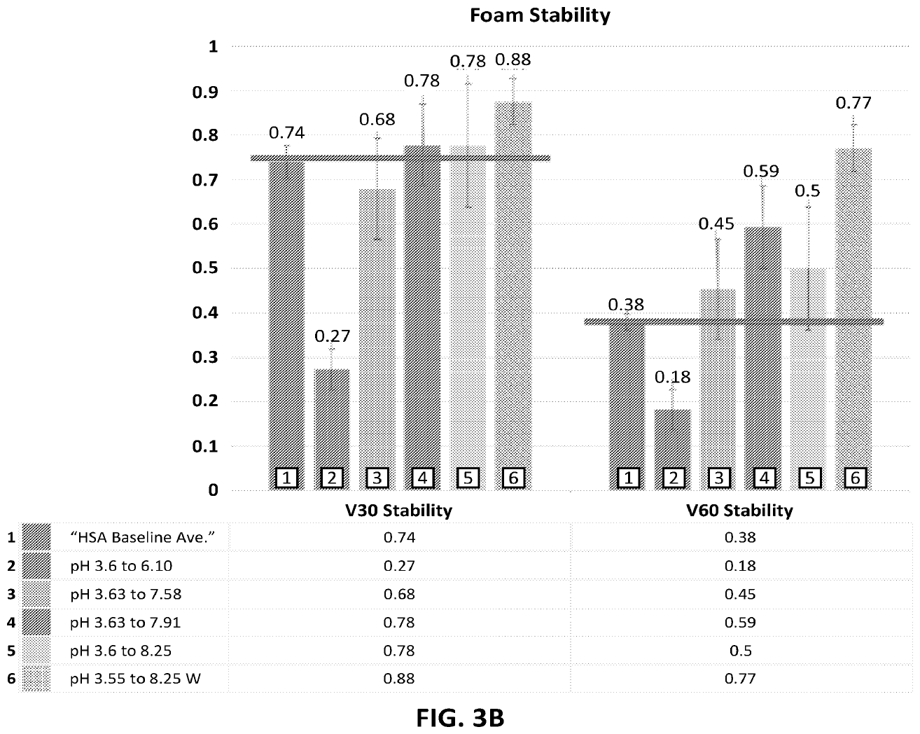

Data acquired from the BSA testing was used to optimize procedures for foaming and pH refolding of HSA. The baseline HSA sample had buffer included, since no buffer-free HSA was available. Refolding the HSA with the new procedure consistently produced a foam with significantly greater stability (TABLE 3 and FIG. 3A) and a relatively low variation in values. These data demonstrated that the HSA buffer significantly reduced the foam stability, and that this was best counteracted by letting the acid equilibrate after mixing in the syringe for 2 to 5 minutes. Any amount of time past that (e.g., 30 minutes) did not provide a justifiable increase in stability for the time commitment. The acidic solution also required that the resting time did not continue for too long; otherwise it became unmanageable to mix with the base. The trends observed for BSA were comparable to HSA, so going forward, BSA can be used for new experimental conditions for initial testing.

The results of pH folding experiments also are plotted in FIG. 3B. For the HSA, pH was decreased to the limit of 3.6 due to HSA buffer composition, and increased to varying levels represented by each bar in FIG. 3B. The horizontal line corresponds to the HSA foam with no pH change. A larger increase in pH resulted in a longer foam stability. Changing the method of foam preparation also affected foam stability. Foams were originally produced by passing the solution through a valve between two 20 mL syringes. Using an immersion blender with a whisk attachment resulted in more stable foams with visibly smaller average bubble size. This method also increases the foam production scale.

TABLE 3
HSA refold stability and equilibration time

| | HSA Refold | HSA Baseline |
|---|---|---|
| 30 minute % stability | 70 | 63 |
| 60 minute % stability | 57 | 17 |

### Example 6—Effect of Lidocaine Hydrochloride on the Stability of Albumin Foam

Further studies were conducted to evaluate the effects of adding lidocaine hydrochloride at a concentration based on solubility limits. The concentration of lidocaine hydrochloride was adjusted to avoid toxicity, using 100 cc of albumin foam for a theoretical 50 kg patient (0.45 mg/mL in a 25 wt % albumin solution). The addition of lidocaine hydrochloride caused a measurable decrease in the pH of the albumin solution, from 6.5 to 4.4. Data were collected using the optimized pH refolding method with syringes, allowing the lidocaine dissolve and equilibrate in the solution before foaming. The observed trends were consistent with the previous findings for HSA.

TABLE 4
Stability of BSA with lidocaine additive

| | Lidocaine | Lidocaine | BSA |
|---|---|---|---|
| | HCl (1) | HCl (2) | Baseline |
| 30 minute % stability | 48 | 22 | 77 |
| 60 minute % stability | 0 | 0 | 4 |

### Example 7—Physiologic Temperature Control and Foam Collapse Baseline at 37□

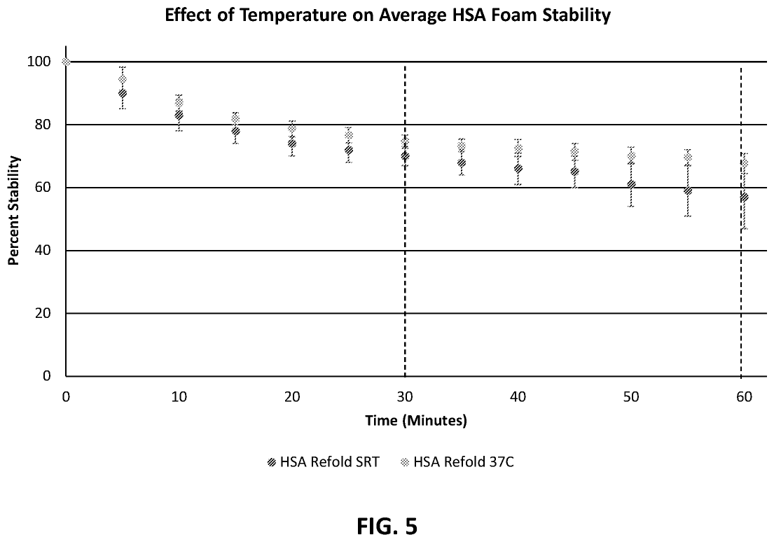

The procedure described above was used for refolding in syringes. Multiple stability tests were run using HSA, and the results are displayed in TABLE 5 and in FIG. 5, with error bars representing the calculated standard deviation. The foam did not decrease in stability at body temperature; the slight increase was not considered significant since the error overlapped for all but one of the data points. The decreased standard deviation for the body temperature average indicated that the constantly held temperature may have prevented sudden changes in the foam and steadied the decay rate.

TABLE 5
Effect of temperature on average HSA foam stability

| | HSA Refold ~22° C. | HSA Refold 37° C. |
|---|---|---|
| 30 minute % stability | 70 | 75 |
| 60 minute % stability | 57 | 68 |

### Example 8—In Vivo Testing

Studies were performed in a porcine model to evaluate how albumin foams behave under physiologic conditions, and how they behave when exposed to ablation energy under physiologic conditions. The studies were done in two phases. In the first phase, foam stability (foam volume/thickness over unit time) at the injection point (ratio of height over diameter) were evaluated. In the second phase, those same parameters were assessed while exposing the foam to ablation energy (e.g., microwave ablation energy or cryoablation energy) under conditions that mirrored conditions achieved while treating tumors in patients. Standard hydrodissection also was perfumed with normal saline as a comparison. Periodic scans were performed to assess foam stability.

### Phase One Study

The phase one study was conducted to track and test the viscosity and stability of the albumin foam composition. The study was divided into an acute arm and a survival arm, each utilizing one to two 40 kg pigs. The acute arm objective of phase one (acute study) was done to evaluate foam stability (foam volume/thickness over unit of time) and viscosity at the injection point. The phase two objective (2 week post-procedure survival) was to assess those same parameters while exposing the foam to ablation energy under conditions that would mirror treating tumors in patients.

In the acute arm of the phase one study, a 40 kg pig was fasted, sedated, and intubated. The pig underwent a baseline CT scan and then, under ultrasound guidance, the albumin foam or normal saline (at a similar location for an internal control) were injected first into the peritoneum, then the chest, and then the retroperitoneum with CT scans performed subsequently, as set forth in TABLE 6. Both the saline and the foam were injected into the perihepatic space, the perinephric space, or the peripheral space. Each injection was done in a physically different location but in the same space (e.g., the right perinephric space vs. the left perinephric space, or the upper right perihepatic space vs. the lower right perihepatic space). The CT scanning tracked the migration and breakdown of the foam. At the end of the series of CT scans, the pig was euthanized and subjected to a gross necropsy to visualize the three target areas.

TABLE 6
Phase 1 protocol - acute arm
CT Scan Time Table

| | | | Time |
|---|---|---|---|
| | Action | Scan | (min) |
| 1. | | Scout scan -WO | |
| 2. | Image-guided Yueh needle placement | | |
| | | Verify access -CE | |
| 3. | Inject foam or saline laced with | p⁰ -WO | 0:00 |
| | iodinated contrast at p locations | | |
| | Inject foam or saline laced with | pl⁰, p¹ | 10:00 |
| | iodinated contrast at pl locations | | |
| | Inject foam or saline laced with | r⁰, pl¹, p² | 20:00 |
| | iodinated contrast at r locations | | |
| | | r¹, pl², p³ | 30:00 |
| | | r², pl³, p⁴ | 40:00 |
| | | r³, pl⁴, p⁵ | 50:00 |
| | | r⁴, pl⁵, p⁶ | 60:00 |
| | | r⁵, pl⁶, p⁷ | 70:00 |
| | | r⁶, pl⁷, p⁸ -CE | 80:00 |
| 4. | Complete protocol, euthanize, | | |
| | necropsy | | |

p, peritoneal;
pl, pleura;
r, retroperitoneal;
WO, without contrast,
CE, contrast enhanced

Study 1: Foam stability in different body spaces. 20 cc of pH-modulated foam mixed with room air was injected into the peritoneal space, the retroperitoneal space, or the pleural space of a 40 kg pig. 20 cc of saline laced with contrast was used as a control. CT scans were taken every 10 minutes for measurement purposes. Results for injection into the peritoneal and retroperitoneal spaces are shown in TABLES 7A and 7B. When injected into the pleural space, the foam dissipated and was not measurable at time 0. This may have been due to the presence of an oligolamellar surfactant; differential chest pressures also may have played a role.

TABLE 7A
In vivo stability studies - peritoneum (perihepatic injection)

| | Foam volume cm³ | Foam length mm | Foam width mm |
|---|---|---|---|
| Time 0 | 30.1 | 46 | 18.1 |
| Time 10 | 28.6 | 47.1 | 16 |
| Time 20 | 28.1 | 47.1 | 14.8 |
| Time 30 | 30.5 | 47.9 | 16 |
| Time 40 | 27.9 | 46.2 | 15 |
| Time 50 | 28.8 | 47.1 | 14.8 |
| Time 60 | 28.8 | 47.1 | 14.1 |
| % change | −4.51 | 2.34 | −28.37 |

TABLE 7B
In vivo stability studies - retroperitoneum (perinephric injection)

| | Foam volume cm³ | Foam length mm | Foam width mm |
|---|---|---|---|
| Time 0 | 28.9 | 54.1 | 21.1 |
| Time 10 | 26.3 | 52.6 | 19.5 |
| Time 20 | 24.7 | 52 | 20.1 |
| Time 30 | 24.6 | 52 | 19.6 |
| Time 40 | 24.4 | 51.1 | 18.9 |
| Time 50 | 24.1 | 50.8 | 18.6 |
| Time 60 | 24.4 | 50.7 | 18.9 |
| % change | −18.44 | −6.71 | −11.64 |

### Phase Two Study

In the second (survival) phase of the study, a 40 kg pig was sedated and intubated before being subjected to the protocol set forth in TABLE 8.

TABLE 8
Phase 2 protocol
Protocol

| | Action | Scan |
|---|---|---|
| 1. | Scout imaging for planning | CT scan (CE, if needed) |
| 2. | Sterile prep | |
| 3. | Image-guided Yueh needle placement | CT WO and/or Ultrasound |
| 4. | Inject foam or saline laced with | CT WO scan |
| | iodinated contrast at access locations | |
| 5. | Image-guided probe placement | CT WO and/or Ultrasound |
| 6. | Pre ablation assessment | CT WO scan |
| 7. | Ablation of the liver | Periodic CT WO scan |
| 8. | Probe removal | during ablation |
| 9. | Assess ablation zone/evaluate | CT CE scan |
| | for bleeding/complications | |

The pig was then allowed to recover. About two weeks later, the pig underwent a another CT scan to evaluate the consequences of the foam use during the ablation procedure, as compared to a normal saline (laced with iodinated contrast) control, which is standard for hydrodissection technique. After the final CT scan, the pig was euthanized and a gross necropsy was performed.

The CT data sets from each of phase of the study were evaluated using Visage software. This allowed for various measurements and calculations to be made, including foam thickness to spread ratio (depth:width) at the site of injection, as well as foam volume calculations and free air calculations to measure foam degradation/stability. The data were compared across each of the spaces injected (peritoneum, retroperitoneum, and pleura) and across phases to compare stability without and with exposure to ablation energy.

Study 2A: Foam stability when exposed to microwave energy. 20 cc of pH-modulated foam mixed with room air was injected into the perihepatic space of a 40 kg pig. A microwave probe was placed 9 mm from the edge of the hepatic capsule and injected foam. The control was the same but without the foam. Microwave ablation was conducted at 65 W for 5 minutes. Foam measurements were taken by CT scan every minute during ablation, and one time post-ablation.

TABLE 9A
In vivo stability studies - microwave energy

| | Foam volume cm³ | Foam length mm | Foam width mm |
|---|---|---|---|
| Injection* | 23.6 | 46.1 | 15.2 |
| Ablation^{¶} | 23.6 | 47.8 | 12.6 |
| 1 Min^{§} | 22.9 | 46.3 | 10.5 |
| 2 Min | 24.8 | 45.9 | 12.1 |
| 3 Min | 22.8 | 47.7 | 12.4 |
| 4 Min | 23.4 | 45.7 | 13.7 |
| 5 Min | 23.7 | 47.9 | 12.4 |
| % change | 0.42 | 3.76 | −22.58 |

*Injection refers to the first foam measurement scan, right after injection.
^{¶}Ablation refers to the measurement after the needle was placed, but before the ablation energy was activated.
^{§}Subsequent timed measurements refer to the foam 1 minute after probe activation (application of energy into the system), 2 minutes after probe activation, etc.

Study 2B: Foam stability when exposed to cryoablation energy. 20 cc of pH-modulated foam mixed with room air was injected into the perihepatic space of a 40 kg pig. A microwave probe was placed 5 mm from the edge of the hepatic capsule and injected foam. The control was the same but without the foam. Cryoablation was performed with a 10 minute freeze, 5 minute thaw and 10 minute refreeze cycle. Foam measurements were taken by CT scan every 2 minutes during the cryoablation procedure and once post-ablation.

TABLE 9B
In vivo stability studies - cryoablation energy

| | Foam volume cm³ | Foam length mm | Foam width mm |
|---|---|---|---|
| Injection | 28 | 47.8 | 16.3 |
| Ablation | 26.8 | 41.3 | 15.7 |
| Refreeze 2 Min | 26.1 | 42 | 17.3 |
| Refreeze 4 Min | 27.8 | 41.8 | 17 |
| Refreeze 6 Min | 28.1 | 42 | 16.7 |
| Refreeze 8 Min | 27.8 | 41.7 | 16 |
| Refreeze 10 Min | 28.1 | 40.7 | 16.7 |
| % change | 7.12 | −3.19 | −3.59 |

### Example 9—In Vitro Testing System

An in vitro testing system was developed to approximate physiologic conditions. The system and procedure utilized beef or porcine liver, with testing for compatibility. The system included a modified graduated cylinder having a side port to allow infusion of foam between two discs of liver cut to fit the cylinder, such that tissue displacement could be measured over a period of time. Normal saline was used to “lubricate” the discs within the system. Studies were performed at or near physiological temperatures, using the characterization method of foam column collapse under weight in the cylinder. The top piece of liver had a known mass and could slide freely, in order to measure the stability of the foam as a function of time in contact with biologic tissue, and to demonstrate displacement of tissue by the foam. Additional studies utilized a larger cross-section of bovine or porcine liver slices within a free-standing container to allow measurement of (1) the displacement of the tissue with foam injection over a larger area, and (2) foam placement vs. lateral spread.

### Example 10—Insulative Properties of Foam

In vitro studies were conducted to determine whether the albumin foam acted as an insulator due to the large volume of air trapped within. A transparent gel was generated and cast within a clear container to enable visual observations. Both agarose and polyacrylamide gels were tested to determine the best chemical composition for these studies. The gel was embedded with thermochromic dyes that switch colors and specific temperatures. Specifically, a dye that changes from blue to clear at 45° C. (the temperature at which tissue damage occurs if sustained for one hour) and a dye that changes from red to yellow at 65° C. were included in the gel matrix. The foam was injected into the gel to hold the foam in place. Ablation was applied and changes in color were detected visually. If the foam did not provide sufficient insulative properties, the surrounding transparent gel changed color.

### Example 11—Expanded In Vivo Studies

The in vivo studies were adjusted to take into account optimization of the foam. In some cases, for example, foam additives that may improve desirable characteristics or add beneficial function to the foam were included in the foam compositions. One such additive was thrombin, which has the ability to decrease the risk of bleeding after percutaneous ablation due to its function in promoting blood coagulation and attenuating hemorrhage. In some cases, additional thrombin-containing foam was injected into the access tract following biopsy (which occurs concurrently with most ablation procedures). The clinical utility of the innocuous, hemostatic foam was further extended for use in vascular embolization, a procedure routinely done to stop internal hemorrhage following trauma, spontaneous gastrointestinal bleeding, and iatrogenic bleeding complications; to thrombose arteriovenous malformations; to thrombose aneurysms and pseudoaneurysms; to treat endoleaks (following endovascular aneurysm repair (EVAR) in stented abdominal aortic aneurysms); and in a variety of tumor related catheter-based interventions.

### Example 12—Ablating a Tumor Near the an Organ to Protect Non-Tumor Tissue

A foam provided herein is injected into a mammal having a tumor, such that the foam is placed between the tumor and an adjacent organ or tissue. The amount of foam injected ranges from about 10 mL to about 1000 mL (e.g., about 25 mL to about 250 mL), and the amount injected typically is sufficient to achieve a separation of about 0.25 cm to about 3 cm (e.g., about 0.5 cm to about 2 cm) between the tumor and the adjacent tissue or organ. The tumor is then subjected to ablation energy (either heat or cold). After ablation, CT scanning is used to determine radiographically whether injury to the tissue or organ adjacent to the tumor can be detected. After a period of time after ablation (e.g., two weeks post ablation), the mammal is sacrificed and the area of the tumor and the adjacent tissue or organ are evaluated by necropsy to determine whether any visible changes are observed.

### Example 13—Reducing Post-Biopsy Bleeding

The hemostatic ability of a thrombin-containing foam is evaluated in vivo following placement of a 17-gauge introducer needle into the kidney, liver, spleen, and/or lung, which are biopsy locations with the potential to bleed following use of such an introducer/biopsy device. After introduction of the needle, an albumin foam containing generated with CO₂ and containing thrombin is injected into the site. A large needle (introducer) is placed such that the beveled end of the needle is directed at the target. The inner stylet is removed and the biopsy device is inserted into the shaft of the introducer. After the biopsy sample is removed, 1-5 mL of foam containing thrombin is infused into the introducer as it is retracted from the organ, thus injecting the foam along the tract.

### Example 14—Intravascular Thrombotic Foam

To evaluate the intravascular thrombotic ability of a thrombin-containing foam, test vessels are accessed under ultrasound guidance with a Yueh catheter needle, and a foam is then infused until thrombosis (a stoppage or significant slowing of flow) is evident under ultrasound or x-ray fluoroscopy. The degree of thrombosis is determined by target vessel size, flow rate, and/or flow volume.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for protecting tissue within a mammal, said method comprising:
administering to said mammal a foam composition comprising a mammalian albumin polypeptide and a gas, wherein said administering comprises injecting an amount of said foam composition into said mammal at a site between a target tissue and a non-target tissue, such that said foam physically separates said target tissue from said non-target tissue, and
subjecting said mammal to an ablation procedure directed to said target tissue,
wherein said foam protects said non-target tissue from damage by said ablation procedure.

2-3. (canceled)

4. The method of claim 1, wherein said gas is air or carbon dioxide.

5. (canceled)

6. The method of claim 1, wherein said foam composition further comprises an additive.

7-14. (canceled)

15. A method for embolizing a blood vessel, said method comprising injecting into said blood vessel an amount of a foam composition effective to embolize said blood vessel, wherein said foam comprises a mammalian albumin polypeptide and a gas.

16-28. (canceled)

29. A method for inducing thrombosis at a biopsy site in a tissue of a mammal, said method comprising:
obtaining a tissue sample from a selected location in said mammal, and
administering to said mammal a foam composition comprising a mammalian albumin polypeptide, thrombin, and a gas, wherein said administering comprises injecting an amount of said foam composition into said mammal at said selected location, wherein said foam induces thrombosis at said selected location.

30-32. (canceled)

33. A method for inducing thrombosis in a blood vessel of a mammal, said method comprising injecting into said blood vessel of said mammal a foam composition comprising a mammalian albumin polypeptide, thrombin, and a gas, wherein said injected foam induces thrombosis in said blood vessel.

34-36. (canceled)

37. A foam composition comprising a mammalian albumin polypeptide and a gas, wherein said composition has a stability of at least 50% after 30 minutes in vivo.

38. (canceled)

39. The foam composition of claim 37, wherein said albumin polypeptide is a human albumin polypeptide.

40. The foam composition of claim 37, wherein said gas is air.

41. The foam composition of claim 37, wherein said gas is carbon dioxide.

42. The foam composition of claim 37, wherein said foam composition consists essentially of said mammalian albumin polypeptide and said gas.

43. The foam composition of claim 37, wherein said foam composition further comprises an additive.

44. The foam composition of claim 43, wherein said additive is protamine or lidocaine.

45. The foam composition of claim 43, wherein said additive is a therapeutic agent.

46. The foam composition of claim 45, wherein said therapeutic agent is an antibiotic or a coagulant.

47. The foam composition of claim 46, wherein said therapeutic agent is an antibiotic selected from the group consisting of penicillins, tetracyclines, cephalosporins, quinolones, lincomycins, macrolides, sulfonamides, glycopeptides, aminoglycosides, and carbapenems.

48. The foam composition of claim 46, wherein said therapeutic agent is a coagulant selected from the group consisting of thrombin, clotting factors of the coagulation cascade, zinc, and antifibrinolytic drugs.

49. The foam composition of claim 43, wherein said additive is a contrast agent.

50. The foam composition of claim 49, wherein said contrast agent is an iodine-based contrast agent.

51. The foam composition of claim 50, wherein said iodine-based contrast agent is iohexol.
